SEQUENCE LISTING (2 sequences)

SEQ ID NO: 1 - DNA, 20 bp, Artificial Sequence
  TRIM8 - Forward Primer
cctatctgcctgcacgtttt

SEQ ID NO: 2 - DNA, 20 bp, Artificial Sequence
  TRIM8 - Reverse Primer
gttgtaggcctggttgcact

(19) United States
(12) Patent Application Publication
(10) Pub. No.: US 2021/0052593 A1
(43) Pub. Date: Feb. 25, 2021

(54) Compositions and Methods for the Treatment of Macrophage Activation Syndrome

(71) Applicant: Children's Hospital Medical Center, Cincinnati, OH (US)

(72) Inventors: Alexei Grom, Cincinnati, OH (US); Grant Schulert, Cincinnati, OH (US)

(21) Appl. No.: 16/977,874

(22) PCT Filed: Mar. 8, 2019
(86) PCT No.: PCT/US19/21290

§ 371 Date: Sep. 3, 2020

Related U.S. Application Data
(60) Provisional application No. 62/640,638, filed on Mar. 9, 2018.

(51) Int. Cl. A61K 31/52; G01N 33/53

(52) U.S. Cl. CPC A61K 31/52; G01N 33/5308

(57) ABSTRACT

Described are methods for the treatment of individuals having or as risk for having Macrophage Activation Syndrome (MAS). The disclosed methods may include the steps of detecting a tripartite motif 8 (TRIM8) protein level or a tripartite motif 8 (TRIM8) mRNA level in abiological sample obtained from an individual, and providing a treatment to said individual based on the level of TRIM8 protein and/or mRNA detected. Individuals identified as being high risk for MAS may be treated with an immunosuppressive therapy as disclosed herein.

### CROSS-REFERENCE TO RELATED APPLICATIONS

This application claims priority to and benefit of U.S. Provisional Application Ser. No. U.S. 62/640,638, to Grom et al, entitled “TRIM8 serves as a biomarker and therapeutic target in systemic juvenile idiopathic arthritis and macrophage activation syndrome,” filed Mar. 9, 2018, the contents of which are incorporated in their entirety for all purposes.

### STATEMENT REGARDING FEDERALLY-SPONSORED RESEARCH

This invention was made with U.S. government support under NIH grant AR059049 The U.S. government has certain rights in this invention.

### BACKGROUND

Macrophage activation syndrome (MAS) is a life-threatening episode of hyperinflammation driven by excessive activation and expansion of T cells and hemophagocytic macrophages producing proinflammatory cytokines and a potentially fatal complication of rheumatic diseases that is caused by excessive activation and expansion of T lymphocytes (predominantly CD8+) and of macrophages exhibiting hemophagocytic activity [1-7]. These events lead to overproduction of cytokines and a hyperinflammatory state associated with cytopenias, liver dysfunction and coagulopathy resembling disseminated intravascular coagulation. Bone marrow (BM) biopsy helps establish the diagnosis since the presence of hemophagocytic macrophages in the BM is the pathognomonic feature of MAS. MAS remains a major cause of mortality in rheumatology with reported death rates as high as 30% [6-7]. Although MAS has been associated with most rheumatic diseases, in pediatrics, it is by far most common in systemic juvenile idiopathic arthritis (SJIA) [1-7]. MAS bears close clinical resemblance to hemophagocytic lymphohistiocytosis (HLH), a constellation of life-threatening histiocytic disorders associated with both primary genetic (pHLH) and secondary acquired causes (sHLH). Primary HLH is not a single disease, but rather a group of rare autosomal recessive immune disorders linked to various genetic defects all affecting the perforin-mediated cytolytic pathway. Many rheumatologists view MAS in SJIA as a distinct form of sHLH. Consistent with this view, up to 40% of MAS/SJIA patients carry hypomorphic mutations in pHLH genes. MAS remains a major source of mortality in Rheumatology, and as such, is an important target for new treatments. As such, the instant disclosure seeks to address one or more of the aforementioned needs in the art.

### BRIEF SUMMARY

Described are methods for the treatment of individuals having or as risk for having Macrophage Activation Syndrome (MAS). The disclosed methods may include the steps of detecting a tripartite motif 8 (TRIM8) protein level or a tripartite motif 8 (TRIM8) mRNA level in a biological sample obtained from an individual, and providing a treatment to said individual based on the level of TRIM8 protein and/or mRNA detected. Individuals identified as being high risk for MAS may be treated with an immunosuppressive therapy as disclosed herein.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. Correlation between serum levels of ferritin (X-axis) and INF-γ, INF induced chemokines (CXCL9/10), and TNF in patients with active MAS at sampling (grey triangles) and in patients with active SJIA without MAS at sampling (black triangles).

FIG. 2. STAT1 phosphorylation within peripheral monocytes upon INF-γ stimulation in vitro. Monocytes were left unstimulated or stimulated with 100 units/mL of IFN-γ for 30 minutes to assess the degree of STAT1 phosphorylation by intracellular flow cytometry. †=p<0.05, ††=p<0.01.

FIG. 3. TRIM8 expression in freshly isolated peripheral blood monocytes. Compared to healthy controls, TRIM8 was one of the most highly over expressed genes in SJIA patients (7.2 fold on average, fdr<10⁻²³). Grey triangles indicate patients with overt MAS.

FIG. 4. Unsupervised clustering of single cell RNA sequencing from three healthy donors and a patient with new-onset systemic JIA at the early stages of MAS. Clustering was based upon previously identified genes contributing to variability of normal bone marrow macrophages, excluding genes with low expression levels. Cells are shown left to right and separated into clusters as shown. The box indicates the group of cells from SJIA with distinct gene expression signature including marked upregulation of genes involved in phagosome activity as well as in the IFN induced cascade.

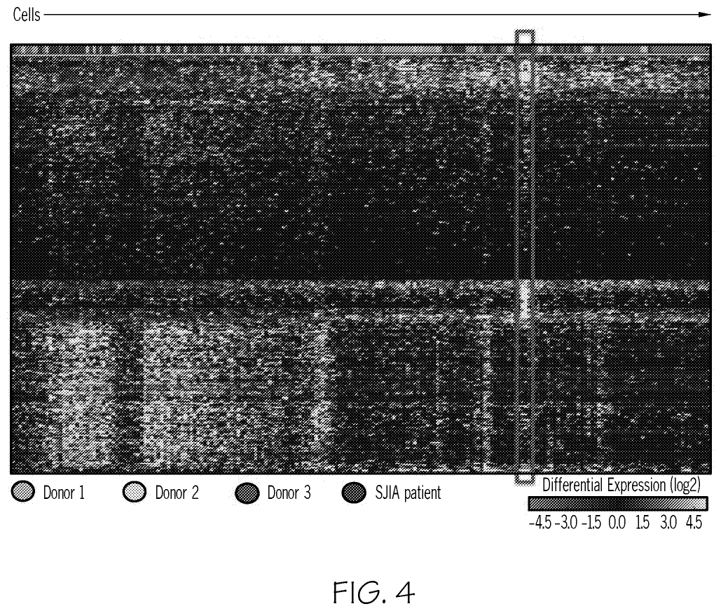

FIG. 5. TRIM8 knockdown via siRNAs in THP-1 cells and monocyte-derived macrophages (MDM) led to decreased production of CLCL9 and CXCL11 in response to stimulation with INFγ in vitro. THP-1 cells or MDMs were treated with either ON-TARGET plus non-targeting siRNA or specific siRNA against TRIM8 will be left untreated in RPMI with 10% FCS or stimulated with IFN-γ (200 u/ml). CXCL9/CXCL10 expression was assessed by RT-PCR at 12 hours.

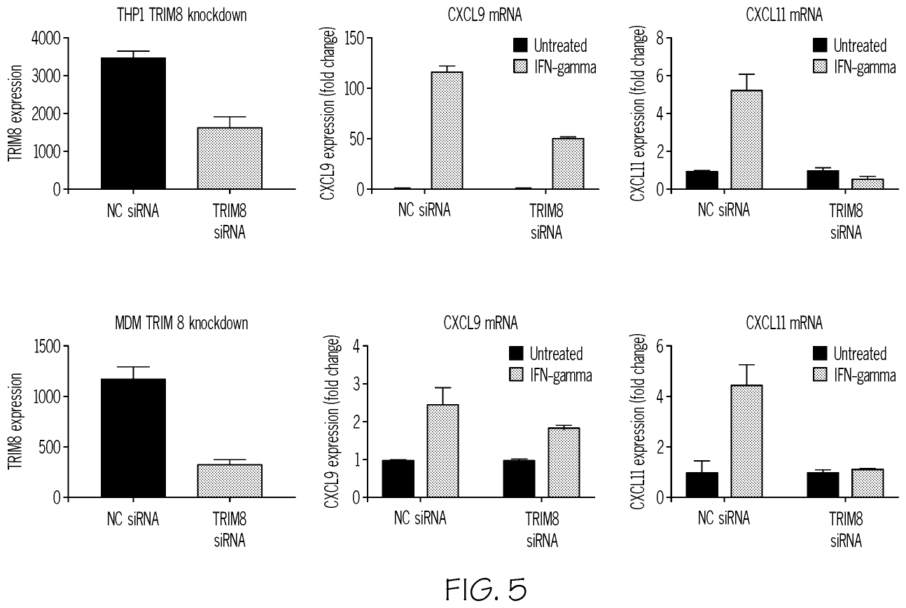

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal (mammal) that is the object of treatment, observation and/or assessment as described herein. Generally, the term refers to a human patient, but the methods and compositions may be equally applicable to non-human subjects such as other mammals. In some embodiments, the terms refer to humans. In further embodiments, the terms may refer to children, for example, individual that are pre-pubescent.

The terms “treating” or “treatment” of a disease includes inhibiting the disease (slowing or arresting or partially arresting its development), providing relief from the symptoms or side effects of the disease (including palliative treatment), and/or relieving the disease (causing regression of the disease).

The phrase “therapeutically effective amount” means an amount of a compound or a combination of compounds that partially or fully ameliorates, attenuates or eliminates one or more of the symptoms of a particular disease or condition or lessens, modifies, or delays the onset of one or more of the symptoms of a particular disease or condition. Such amount can be administered as a single dosage or can be administered according to a regimen, whereby it is effective. Repeated administration may be needed to achieve a desired result (e.g., treatment of the disease and/or condition).

The term “immunomodulator” refers to a medicine used to help regulate or normalize the immune system by inducing, enhancing, suppressing or weakening an immune response in a patient.

The active agent may form salts, which are also within the scope of the preferred embodiments. Reference to a compound of the active agent herein is understood to include reference to salts thereof, unless otherwise indicated. The term “salt(s)”, as employed herein, denotes acidic and/or basic salts formed with inorganic and/or organic acids and bases. In addition, when an active agent contains both a basic moiety, such as, but not limited to an amine or a pyridine or imidazole ring, and an acidic moiety, such as, but not limited to a carboxylic acid, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps, which may be employed during preparation. Salts of the compounds of the active agent may be formed, for example, by reacting a compound of the active agent with an amount of acid or base, such as an equivalent amount, in a medium such as one in which the salt precipitates or in an aqueous medium followed by lyophilization. When the compounds are in the forms of salts, they may comprise pharmaceutically acceptable salts. Such salts may include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like.

“Sequence identity” as used herein indicates a nucleic acid sequence that has the same nucleic acid sequence as a reference sequence, or has a specified percentage of nucleotides that are the same at the corresponding location within a reference sequence when the two sequences are optimally aligned. For example a nucleic acid sequence may have at least 50%, 60%, 70%, 75%, 80%, 85%, 90%, 95%, 96%, 97%, 98%, 99%, or 100% identity to the reference nucleic acid sequence. The length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

As discussed above, macrophage activation syndrome (MAS) is a life-threatening episode of hyperinflammmation driven by excessive activation and expansion of T cells and hemophagocytic macrophages producing proinflammatory cytokines, and has been reported in association with almost any rheumatic disease, but it is by far most common in systemic juvenile idiopathic arthritis (SJIA). MAS bears close clinical resemblance to hemophagocytic lymphohistiocytosis (HLH), a constellation of life-threatening histiocytic disorders associated with both primary genetic (pHLH) and secondary acquired causes (sHLH). Primary HLH is not a single disease, but rather a group of rare autosomal recessive immune disorders linked to various genetic defects all affecting the perforin-mediated cytolytic pathway. Many rheumatologists view MAS in SJIA as a distinct form of sHLH. Consistent with this view, up to 40% of MAS/SJIA patients carry hypomorphic mutations in pHLH genes. MAS remains a major source of mortality in Rheumatology, such that advances in the treatment of MAS are needed.

A ‘cytokine storm’ is the final pathophysiologic pathway in both MAS and HLH. Applicant suggests that blocking various cytokines could be an attractive therapeutic strategy. Animal models and translational studies in pHLH patients support IFN-γ blockade as novel therapy for pHLH, and a Phase III trial of an anti-IFNγ antibody in pHLH is in progress. However, the role of IFN-γ in SJIA-associated MAS has not yet been fully determined.

Interestingly, IFN-γ does not play a major role in the pathogenesis of SJIA itself, but in preliminary studies, Applicant has found that the development of MAS features in patients with SJIA paralleled activation of the IFN-induced signaling pathway in circulating monocytes and bone marrow (BM) hemophagocytic macrophages. In fact, activation of this pathway distinguished acute MAS versus a conventional flare of SJIA. Furthermore, monocytes from SJIA patients exhibited hyper-responsiveness to IFN-γ in vitro that was further exaggerated by IL-1 and IL-6 inhibiting biologics, a finding that could explain unexpectedly high rates of MAS in SJIA treated with these agents.

Applicant found that single cell RNA-Seq data from hemophagocytic BM macrophages identified several proteins that could contribute to the exaggerated responsiveness of macrophages to IFN-γ, including tripartite motif 8 protein (TRIM8) that has been recently implicated in positive regulation of IFN-signaling. Based on these observations, Applicant suggests that monocyte/macrophage 1FNγ hyper-responsiveness and excessive activation of the IFN-induced signaling pathway in monocytes/macrophages play a pivotal role in MAS pathogenesis, and may be targeted therapeutically.

In one aspect, a method of identifying and treating an individual as having or being high risk for Macrophage Activation Syndrome (MAS) is disclosed. In this aspect, the method may comprise the steps of detecting a tripartite motif 8 (TRIM8) protein level or a tripartite motif 8 (TRIM8) mRNA level in a biological sample obtained from said individual; and providing a treatment to said individual.

In one aspect, if the TRIM8 protein level or the mRNA level is increased compared to a control value, the individual is identified as being high risk for MAS. In this aspect, the treatment may comprise administering an immunosuppressive therapy as described here. In one aspect, if the TRIM8 protein or mRNA level is not increased or is the same as compared to a control value, the individual is not identified as being at high risk for MAS and the treatment does not comprise administration with an immunosuppressive therapy as described herein.

In one aspect, the increase in TRIM8 protein or TRIM8 mRNA level is considered to be increased when the level of TRIM8 protein or TRIM8 mRNA in the biological sample is two standard deviations above and below the mean of levels for a healthy individual. TRIM8 protein levels may be assessed using any method known in the art, for example, ELISA. TRIM8 RNA levels may be assessed using any method known in the art, for example, RT-PCR. In one aspect, the increase in TRIM8 protein or TRIM8 mRNA level is considered to be increased when the level of TRIM8 protein or TRIM8 mRNA in the biological sample exceeds the level of TRIM8 protein or TRIM8 mRNA in a sample from a healthy control subject by 5%, 10%, 20%, 30%, 40%, 50%, 60%, 70%, 80%, 90%, 100%, or more than 100%. In such aspect, the individual may be considered as having or at high risk for having MAS and administration of an immunosuppressive therapy as described herein is indicated.

In one aspect, disclosed is a method for identifying and treating an individual as having or being high risk for Macrophage Activation Syndrome (MAS), in an individual comprising detecting the specific binding of a tripartite motif 8 (TRIM8) protein with an antibody specific for TRIM8 in a sample or in situ which may include the steps of:

a) bringing a sample or a specific body part or body area suspected to contain TRIM8 protein into contact with a TRIM8 specific antibody, which specifically binds to TRIM8;

b) allowing the TRIM 8 antibody to bind to TRIM8;

c) detecting the binding of the antibody and determining the amount of TRIM8 in the sample;

d) comparing the amount of TRIM8 in the sample of the individual to the amount in the sample of a healthy patient (not diagnosed with an infectious disease or viral infection or suspected of having MAS or SIJA) (the “control value”);

wherein an increase in the amount of TRIM8 in the sample compared to a control value indicates that said patient is likely to have, or is at high risk for, developing Macrophage Activation Syndrome (MAS).

In one aspect, the individual may be diagnosed or suspected of having systemic juvenile idiopathic arthritis (SJIA). In a further aspect, the individual may be diagnosed with a viral illness, for example, H1N1, Dengue fever, adenovirus, EBV, CMV, or a combination thereof. In this aspect, a determination of elevated TRIM8 protein or mRNA compared to a control value in said individual may indicate that the individual is at increased risk of developing MAS. In such instance where the individual has an increased risk of developing MAS, an agent as described herein may be administered. The administration may comprise an increased dose compared to a recommended dose, an increase in the frequency as compared to a recommended frequency of administration, or a combination thereof. Viral illnesses may include, for example, Influenza, Bronchitis, Gastroenteritis, Croup, RSV, noroviral infection, rotavirus infection, rabies virus infection, West Nile virus infection, Zika virus infection, rubella virus infection.

In one aspect, the biological sample may be blood. In one aspect, the biological sample may be monocytes from an individual of interest. The sample may be a non-diluted or diluted biological fluid, such as, without being restricted thereto, serum, urine, tear, saliva, bile, sweat, exhalation or expiration, sputum, bronchoalveolar fluid, sebum, cellular, gland, mucosa or tissue secretion, biopsy, homogenized tissue.

In one aspect, the immunosuppressive therapy may be selected from a JAK/STAT inhibitor, an anti-interferon gamma agent, an increased dosage of an existing immunosuppressive regime, or a combination thereof.

In one aspect, the immunosuppressive therapy may be an immunomodulator selected from tofacitimib, ruxolitinib, baricitinib, and oclacitinib. Other agents include, but are not limited to JAK inhibitor is selected from one or more of the group consisting of: INCB0391 10, AZD1480, fedratinib, AT9283, AG-490, momelotinib, WP1066, TG101209, gandotinib, NVP-BSK805, AZ 960, CEP-33779, Pacritinib, WHI-P154, XL019, S-Ruxolitinib, ZM 39923, Decernotinib, Cerdulatinib, filgotinib, FLLL32, BMS-91 1543, peficitinib, GLPG0634, GLPG0634 analogue, Go6976, curcumol, cucurbitacin, lestaurtinib, upadacitinib, CHZ868, Solcitinib (GSK 2586184), NS-018; etanercept, infliximab, adalimumab, tocilizumab, rituximab, ofatumumab, belimumab, epratuzumab, abatacept, golimumab, certolizumab pegol, sifalimumab, anakinra, canakinumab, rilonacept, pf-04965842, or a derivative thereof; or pharmaceutically acceptable salt thereof. Other JAK inhibitors may include those described in WO2017143014, “Jak inhibitors and uses thereof” published Aug. 24, 2017. In some aspects, the immunomodulator is a JAK/STAT modulating compound selected from 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, tofacitinib, TG101348, Janex 1, PF-956980, WHI-P154, ZM-39923, NSC114792, PF-06263276, CEP-33779, AZD-1480, SHR0302, oclacitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, a salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, an enantiomer thereof, a diastereomer thereof, a racemate thereof, a mixture of stereoisomers thereof, and a combination thereof. In some embodiments, the JAK/STAT modulating compound is 5-((2-((4-fluoro-3-methoxy-5-methylphenyl)amino)-5-methylpyrimidin-4-yl)amino)benzo [d]oxazol-2(3H)-one or disodium (5-((2-((4-fluoro-3-methoxy-5-methylphenyl)amino)-5-methylpyrimidin-4-yl)amino)-2-oxobenzo[d]oxazol-3(2H)-yl)methyl phosphate. In some embodiments, the route of administration of the JAK/STAT modulating compound is selected from the group consisting of oral, topical, systemic, subcutaneous, intramuscular, intraperitoneal, transdermal, intravenous injection, and a combination thereof.

In one aspect, the immunosuppressive therapy may be an anti-interferon gamma agents, for example emapalumab, which blocks interferon gamma.

In one aspect, the immunosuppressive therapy may be selected from one or more of azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate. In one aspect, the immunosuppressive therapy may include at least one selected from the group consisting of a JAK1 inhibitor, a JAK2 inhibitor, a JAK3 inhibitor, or a TYK2 inhibitor. In one aspect, the immunosuppressive therapy is tofacitinib.

Where an individual is identified as being high risk for MAS, the immunosuppressive therapy may comprise administration of an amount of immunosuppressive agent that is increased as compared to a recommended dose, such as a dose that is 1.5 times that of the recommended dose, or 2 times that of the recommended dose, or 2.5 times that of the recommended dose, or 3 times that of the recommended dose. The recommended dose may be the mean range of a dose for an individual.

In one aspect, a method of treating an individual having Macrophage Activation Syndrome (MAS) or suspected of being high risk for developing Macrophage Activation Syndrome (MAS) is disclosed, comprising administering an immunosuppressive therapy selected from a JAK/STAT inhibitor, an anti-interferon gamma agent, or a combination thereof. The individual may be diagnosed with systemic juvenile idiopathic arthritis (SJIA) or a viral illness selected from H1N1, Dengue fever, adenovirus, EBV, CMV, or a combination thereof, wherein a determination of elevated TRIM8 protein or mRNA compared to a control value in said individual indicates that said individual is at increased risk of developing MAS, wherein said individual having an increased risk of developing MAS is administered an agent as described above.

In one aspect, active agents provided herein may be administered in an dosage form selected from intravenous or subcutaneous unit dosage form, oral, parenteral, intravenous, and subcutaneous. In some embodiments, active agents provided herein may be formulated into liquid preparations for, e.g., oral administration. Suitable forms include suspensions, syrups, elixirs, and the like. In some embodiments, unit dosage forms for oral administration include tablets and capsules. Unit dosage forms configured for administration once a day; however, in certain embodiments it may be desirable to configure the unit dosage form for administration twice a day, or more.

Methods for determining TRIM8 protein levels: Standard methods known in the art, in particular, the ELISA method, may be used to detect TRIM8 protein levels in an individual. Antibodies used in these assays may be monoclonal or polyclonal, and may be of any type such as IgG, IgM, IgA, IgD and IgE. Antibodies may be produced by immunizing animals such as rats, mice, and rabbits. The antigen used for immunization may be isolated from the samples or synthesized by recombinant protein technology. The method may include the use of molecular complexes comprising a TRIM8 specific antibody and a label. Examples of detectable substances include various enzymes, prosthetic groups, fluorescent materials, luminescent materials, bioluminescent materials, and radioactive materials. Examples of suitable enzymes include horseradish peroxidase, alkaline phosphatase, 0-galactosidase, or acetylcholinesterase; examples of suitable prosthetic group complexes include streptavidin/biotin and avidin/biotin; examples of suitable fluorescent materials include umbelliferones, fluoresceins, fluorescein isothiocyanate, rhodamines, dichlorotriazinylamine fluorescein, dansyl chloride, phycoerythrins, Alexa Fluor 647, Alexa Fluor 680, DilC19(3), Rhodamine Red-X, Alexa Fluor 660, Alexa Fluor 546, Texas Red, YOYO-1+DNA, tetramethylrhodamine, Alexa Fluor 594, BODIPY FL, Alexa Fluor 488, Fluorescein, BODIPY TR, BODIPY TMR, carboxy SNARF-1, FM 1-43, Fura-2, Indo-1, Cascade Blue, NBD, DAM, Alexa Fluor 350, aminomethylcoumarin, Lucifer yellow, Propidium iodide, or dansylamide; an example of a luminescent material includes luminal; examples of bioluminescent materials include green fluorescent proteins, modified green fluorescent proteins, luciferase, luciferin, and aequorin, and examples of suitable radioactive material include 125I, 131I, 35S or 3H. The immunoassays will typically comprise incubating a sample, such as a biological fluid, a tissue extract, freshly harvested cells, or lysates of cells, in the presence of a detectably labeled TRIM8 specific antibody fragments thereof, and detecting the bound antibody by any of a number of techniques well-known in the art. One way of measuring the level of TRIM8 with the antibody is by enzyme immunoassay (EIA) such as an enzyme-linked immunosorbent assay (ELISA)

Exemplary method for determining TRIM8 expression. TRIM8 expression may be assessed using RT-PCR as described in [79], and gene expression may be normalized against GAPDH. The following TRIM8 primers may be used: “Forward” 5′CCTATCTGCCTGCACGTTTT-3′ (SEQ ID NO: 1); “Reverse” 5′-GTTGTAGGCCTGGTTGCACT-3′ (SEQ ID NO: 2).

Exemplary method for monocyte isolation. Fresh whole blood is collected in ACD solution A vacutainer tubes (Becton Dickinson, Franklin Lakes, N.J.). CD14+ monocytes are immediately separated within 15-20 minutes of collection using whole blood CD14 microbeads and the AutoMACSPro (Miltenyi Biotec, San Diego, Calif.).

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

MAS in Rheumatology. Macrophage activation syndrome (MAS) is a potentially fatal complication of rheumatic diseases that is caused by excessive activation and expansion of T lymphocytes (predominantly CD8+) and of macrophages exhibiting hemophagocytic activity [1-7]. These events lead to overproduction of cytokines and a hyperinflammatory state associated with cytopenias, liver dysfunction and coagulopathy resembling disseminated intravascular coagulation. Bone marrow (BM) biopsy helps establish the diagnosis since the presence of hemophagocytic macrophages in the BM is the pathognomonic feature of MAS. MAS remains a major cause of mortality in rheumatology with reported death rates as high as 30% [6-7]. Although MAS has been associated with most rheumatic diseases, in pediatrics, it is by far most common in systemic juvenile idiopathic arthritis (SJIA) [1-7]. The pathophysiology of SJIA seems to be driven by continuous activation of innate immune pathways leading to dysregulated production of proinflammatory cytokines. Therefore, many pediatric rheumatologists view SJIA as an autoinflammatory disorder rather than a classic autoimmune disease [8-10]. IL-1P [11-13] and IL-6 [14-16] have been implicated as essential cytokines in the pathogenesis of this condition. Indeed, large phase III trials with the IL-1P inhibitor canakinumab [13] and the IL-6 receptor inhibitor tocilizumab [16] showed that 60-70% of SJIA patients experience profound SJIA improvement with up to 40% achieving sustained clinical remission of SJIA. Different from what experts had predicted, control of SJIA did not protect against MAS [13,16].

MAS and HLH. MAS bears striking clinical resemblance to hemophagocytic lymphohistiocytosis (HLH) [17]. HLH is further divided into primary (or familial) HLH and secondary HLH (also known as acquired or reactive HLH) [18-21]. Primary HLH (pHLH) is a constellation of rare autosomal recessive immune disorders linked to various genetic defects, all affecting the perforin-mediated cytolytic pathway. In normal physiologic conditions, cytolytic cells (such as cytotoxic CD8 T lymphocytes or NK cells) induce apoptosis of cells infected with viruses or cells undergoing malignant transformation. Even moderate defects in the cytolytic pathway might prolong the survival of target cells and increase immune synapse time, ultimately leading to overproduction of proinflammatory cytokines including IFN-γ [22]. Cytolytic cells can also directly induce apoptosis of overly activated immune cells [23-25]. These observations led to the hypothesis that, in HLH, failure to induce apoptosis of target cells by cytolytic cells leads to persistent expansion of activated T lymphocytes and macrophages and escalated production of cytokines, thus creating a ‘cytokine storm’.

Genetic abnormalities in pHLH. In 30% of patients with pHLH cytolytic dysfunction is due to loss-of-function mutations in the gene encoding perforin (PRF1), a protein utilized by cytolytic cells to induce apoptosis of target cells via granzyme B [26]. When released at the surface interface with the target cell (also called the immune synapse), perforin self-polymerizes, creating pores in the plasma membrane, that enable granzymes to enter the target cell and trigger apoptosis. The genes implicated in other types of pHLH (MUNC13-4, STX11 and STXBP2) encode proteins involved in the transport of granules containing perforin and granzymes to the immune synapse [27-29]. The cytolytic cells in patients with these mutations produce sufficient amounts of perforin, but an impaired ability to release perforin into the immune synapse leads to decreased cytolytic activity. Although mutations in PRF1, MUNC13-4, STX11 and STXBP2 explain the disease in most patients with pHLH,—25% of familial cases are still awaiting molecular definition [18].

Secondary HLH. Secondary HLH (sHLH) can occur at any age and tends to have less severe clinical presentations than in pHLH, but the mortality in this group is still high [18,20]. The emergence of first clinical signs and symptoms in sHLH can usually be linked to an infectious episode or malignancy. Furthermore, the cytolytic pathway abnormalities that occur in this condition are generally considered to be acquired [18,30]. In many of these patients, however, the development of sHLH has been recently linked to heterozygous hypomorphic mutations that confer a partial dominant negative effect on the cytolytic function [31]. These findings make it increasingly difficult to distinguish between primary and secondary HLH.

Cytolytic abnormalities in MAS. Due to striking clinical similarities between MAS and HLH, many view MAS as a distinct form of sHLH. Consistent with this view, profoundly depressed cytolytic function is observed in SJIA patients with MAS [32], although this impairment tends to improve with better control of the activity of the underlying SJIA [33-35] suggesting that background inflammation is a contributing factor. Indeed, IL-6, a pivotal proinflammatory cytokine in SJIA, has been shown to induce defective expression of perforin and decrease NK cell cytotoxic activity [34]. Using whole exome sequencing methodology, Applicant identified novel protein-altering SNPs/indels in the known pHLH-associated genes in 36% of SJIA/MAS patients [36], similar to the percentage of SJIA patients at risk for MAS [37,38]. Such increased “burden” of rare variants in the cytolytic pathway in MAS patients compared to healthy controls has been recently reported by others as well [39] suggesting that as in pHLH, genetic variability in the cytolytic pathway contributes to MAS predisposition. Overall, development of cytolytic dysfunction in MAS appears complex and is influenced by inflammatory activity of the underlying disease and by a genetic component [35].

Cytokine storm in MAS/HLH. MAS remains a major source of mortality in Rheumatology partially due to frequently delayed diagnosis [35]. Treatment includes high dose steroids, cyclosporine and etoposide, all associated with considerable toxicity. Cyclosporine has been associated with pulmonary hypertension and posterior reversible encephalopathy syndrome. Life threatening bone marrow suppression, short-term, and hematologic malignancies later in life are common side effects of etoposide. A ‘cytokine storm’ is the final pathophysiologic pathway in both MAS and HLH and blocking various cytokines could be an attractive therapeutic strategy. Studies of the inflammatory infiltrate in both HLH and MAS demonstrate abundance of IFN-γ producing CD8 T lymphocytes found in close proximity to hemophagocytic macrophages producing TNF, IL-18, and IL-6 [40]. Findings in PRF1 and Rab27a knockout mice, two main models of pHLH, implicate these IFN-γ producing CD8 T lymphocytes as the main driving force of HLH [41,42]. Levels of IFN-γ are elevated in children with pHLH, as are levels of IFN-γ-induced chemokines [43,44]. These observations suggested that IFN-γ could be targeted therapeutically in pHLH; and a phase III trial evaluating this approach in pHLH is underway [ClinicalTrials.gov NCT01818492].

Cytokines in MAS. With growing numbers of biologics targeting cytokines and small molecules inhibiting cytokine signaling pathways such as JAK/STAT inhibitors, the interest in relative significance of various cytokines in MAS is increasing.

IL-1 and IL-6. IL-113 [11-13] and IL-6 [14-16] have been implicated as essential cytokines in SJIA, and adequate control of the underlying disease using biologics neutralizing IL-1 or TL-6 was expected to protect against MAS. The observed MAS rates in the phase III clinical trials of tocilizumab (anti-IL6R antibody) and canakinumab (anti-IL1β antibody) have shown, however, that therapeutic strategies aimed at the inhibition of either IL-1β or IL-6 do not provide protection against MAS even if the underlying SJIA is well controlled [45-47]. One possible conclusion is that neither IL-113 nor IL-6 are the key drivers of MAS. Reports describing successful treatment of MAS with anakinra, the recombinant IL-1 receptor antagonist that blocks activity of both IL-113 and IL-1a, suggest a potential role for IL-1a [48-50]. However, the fact that MAS has been seen in patients treated with rilonacept [51], which also neutralizes IL-1a, makes this possibility less likely. Alternatively, the fact that some SJIA/MAS patients respond to IL-1 blockade while others develop MAS during continuous treatment with IL-1 blocking biologics, suggests some MAS pathophysiologic heterogeneity that needs to be explored.

IL-18 in MAS. Over the last five years, interest in the role of IL-18 in the pathogenesis of SJIA in general, and in MAS in particular, has increased. IL-18 is produced by myeloid and epithelial cells and is best known for amplifying lymphocyte production of IFN-γ. Strikingly high serum levels of IL-18 have been observed in patients with SJIA [52,53], in sharp contrast to only moderately elevated levels of IL-18 seen in other rheumatic diseases [54,55]. Patients with high levels of IL-18 more often have systemic manifestations rather than arthritis as the predominant feature of SJIA [52,53] and also seem to be more likely to develop MAS. The role of IL-18 has been examined in perforin-deficient mice infected with murine CMV. Uncontrolled viral replication in these mice is associated with many features of HLH and MAS including pancytopenia and hemophagocytosis [56]. Administration of synthetic IL-18BP ameliorated liver damage in these mice, but production of proinflammatory cytokines remained high and no change in overall survival was observed [56]. Companionate use of Tadekinig-alfa (recombinant IL18BP) in an SJIA patient with recurrent MAS reduced the severity of MAS episodes, but their frequency remained unchanged.

Role of IFN-γ in SJIA versus MAS. Low IFN-activity in SJIA without MAS. The role of IFN-γ in SJIA-associated MAS has not yet been fully determined. Interestingly, IFN-γ does not seem to be involved in the pathogenesis of SJIA itself. Levels of serum IFN-γ have been reported to be within the normal range in patients with SJIA, independent of disease activity [57]. Gene expression studies have failed to find a prominent IFN-γ-induced signature in the peripheral blood monocytes of children with active SJIA but no clinical features of MAS [58-60]. The absence of IFN-γ activity is not limited to only peripheral blood; the expression of IFN-γ-induced chemokines (CXCL9 and CXCL10) in synovial tissue from SJIA patients is hardly detectable, in contrast to very high levels of these chemokines in tissue from patients with oligoarticular or polyarticular JIA [57].

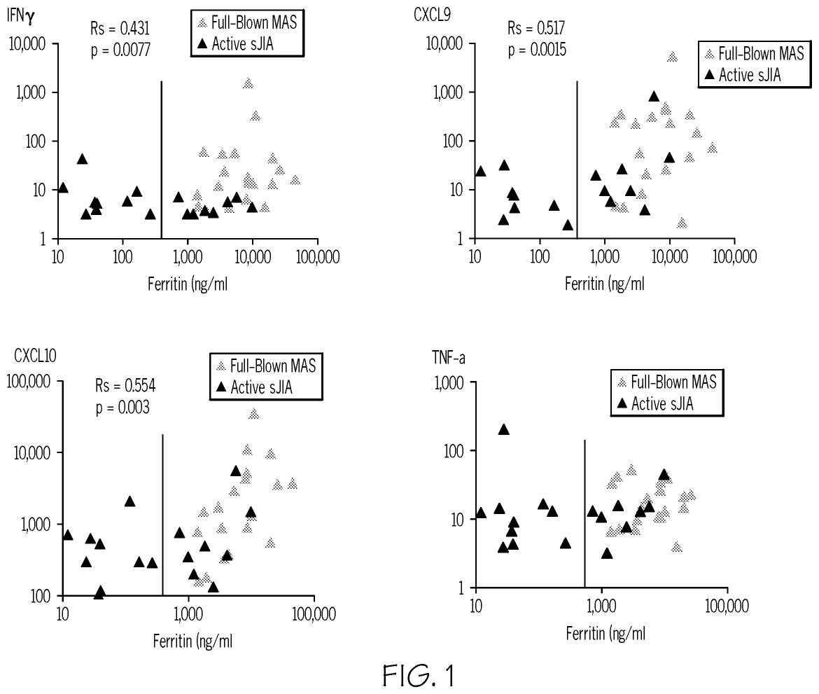

IFN activity biomarkers in SJIA patients with MAS. In contrast to SJIA, preliminary evidence suggests that IFN-γ is essential for the pathogenesis of MAS. Applicant's studies have shown that IFN-γ itself and IFN-γ-induced chemokines increased markedly with the emergence of clinical features of MAS (FIG. 1) and returned to normal ranges after its resolution [61]. Furthermore, such increase was associated with activation of the IFN-induced signaling pathway based on increased STAT1 phosphorylation in freshly isolated unmanipulated monocytes. Binding of IFN-γ to its receptors triggers JAK1/2-STAT1 singling pathway, and the degree of STAT1 phosphorylation reflects the degree of signaling activation [66]. In fact, activation of this pathway distinguished acute MAS from conventional flare of SJIA. In addition, IFN-γ and IFN-induced chemokines strongly correlated with many laboratory features of MAS in patients with clinical features of MAS, but not in patients with a conventional SJIA flare without MAS (FIG. 1). No similar correlations were observed with TNF or IL-6.

IFN-induced signature in BM hemophagocytic macrophages in MAS. Circulating monocytes are recruited into inflammatory sites where in the context of specific cytokine milieu, they mature into different types of resident macrophages. To fully understand the phenotype of macrophages in MAS, Applicant therefore directly examined macrophages at the inflammatory sites such as bone marrow via Single Cell RNA

Cellular sources of IFN-γ. In the inflammatory lesions of MAS there is marked predominance of activated histiocytes and CD8+ T cells producing IFN-γ [40]. A population of CD8+ CD38⁺ CCR7⁻ Granzyme B^{high} cytolyic effector phenotype T cells have recently been identified as the main source of IFN-γ in peripheral circulation in active pHLH. Applicant has observed expansion of this population in MAS (n=2), but not in SJIA patients without MAS regardless of SJIA disease activity (n=12), suggesting that CD8+ CD38⁺ CCR7⁻ T cells might be a prominent source of IFN-γ in MAS as well. Further phenotypic characterization of these cells and identification of clinical patterns associated with the emergence of this population may provide mechanistic clues to understand pathophysiologic reasons for their expansion

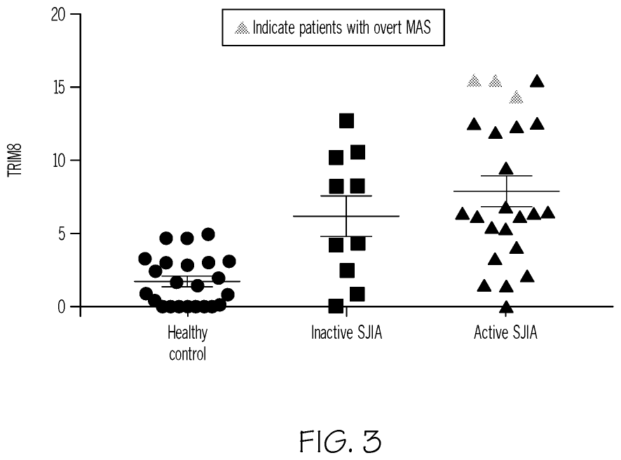

Hyper responsiveness of monocytes to IFN-γ in SJIA. The SJIA without MAS does not seem to be caused by decreased responsiveness to IFN-γ as monocytes from SJIA patients stimulated with IFN-γ in vitro exhibited exaggerated responses to this cytokine. This hyper-responsiveness might be further increased by the biologics used to treat SJIA. The exact mechanisms leading to such hyper-responsiveness remain unknown. Examination of the SC RNA-Seq data from the BM macrophages obtained at the time of acute MAS as well as the RNA-Seq data from freshly isolated peripheral monocytes obtained from SJI/MAS patients with or without MAS (FIG. 3) identified several molecules that could contribute to the exaggerated responsiveness of monocytes and macrophages to IFN-γ. One of those is the tripartite RING-finger protein 8 (TRIM-8) that decreases stability of SOCS1 [62]. SOCS1 is induced by various pro-inflammatory cytokines including IFN-γ and provides negative feedback regulation of IFN-signaling by inhibiting IFN-induced JAK-STAT activation. TRIM-8 was highly overexpressed in peripheral monocytes from SJIA/MAS patients (FIG. 3) as well as in hemophagocytic macrophages in BM of MAS patients. Based on these observations, Applicant hypothesized that TRIM-8 overexpression decreases repression of IFN-induced signaling hypersensitivity to IFN-γ. Consistent with this idea, TRIM8 knockdown via siRNAs in THP1 cells and monocyte-derived macrophages led to decreased production of CXCL9 and CXCL11 in response to stimulation with IFN-γ in vitro.

Although our understanding of MAS pathophysiology has markedly improved over the last decade, the previous approaches used to study this phenomenon have had key limitations. First, most studies examined mixed populations of mononuclear cells almost exclusively in peripheral circulation, which limits the conclusions regarding particular immune subsets. Second, animal models of hemophagocytic syndromes suggest that by the time macrophages acquire their full pathologic phenotype, they leave peripheral circulation and accumulate in tissues such as BM. The few studies that examined macrophages directly at the inflammatory sites in MAS patients, were limited mainly to simple histopathologic descriptions of the inflammatory infiltrates. Recent advances in microfluidics, robotics, amplification chemistries and DNA sequencing technologies provide the ability to isolate, sequence, and quantitate RNA transcripts from single cells. The expertise in the single-cell RNA-Seq technology) provides an exciting opportunity to study both peripheral and BM macrophages from patients with MAS at a single cell level.

Applicant has identified TRIM-8 as a factor likely contributing to hyper-responsiveness of macrophages to IFN-γ in SJIA.

Previous gene expression studies of PBMCs from SJIA patients without apparent MAS failed to demonstrate the IFN-induced signature [59] and immunophenotyping of circulating monocytes revealed a mixed activation phenotype with features of M(LPS+IC) and M(IL-10) macrophages [65]. Applicant's recent data in patients with MAS showed that the development of MAS in SJIA patients associated with a marked increase in the serum levels of IFN-induced chemokines [56]. Additionally, in MAS patients, Applicant observed increased baseline levels of phosphorylated STAT1 in freshly isolated monocytes reflective of increased activation of the IFN-induced signaling pathway. As shown in FIG. 1, activation of this pathway distinguished acute MAS versus conventional flare of SJIA. Combined these data suggest that monitoring this pathway in SJIA may help identify patients in the early stages of MAS before it progresses to its life-threatening stage.

Although very informative, studies of peripheral blood leukocytes have limitations. By the time monocytes acquire their full pathogenic phenotype, they leave peripheral circulation and accumulate in tissues where they differentiate into macrophages. Diagnostic BM biopsies that are frequently performed in MAS provide an excellent source of tissue macrophages. Applicant utilized SC RNA-seq to identify specific gene expression signatures of BM macrophage populations in SJIA and MAS. Cell sources included unused portions of BM aspirates obtained as part of a diagnostic workup for SJIA/MAS. Control samples included BM obtained from patients with FUO and interpreted as normal. Macrophage single cell suspensions were obtained using cell sorting for populations expressing the monocyte and macrophage surface markers CD14 and CD163 while excluding cells expressing the granulocyte/monocyte marker CD15, prior to loading onto the Fluidigm C1 Single-Cell Auto Prep System. Extracted RNA was converted into cDNA and sequenced as a pooled library, and aligned to the human Ensembl transcriptome as described. Three independent control samples yielded 180 single cells which passed quality-control filtering. While there was substantial inter-individual variability, using principle component analysis and Iterative Clustering and Guide-gene Selection, a core set of macrophage population control clusters, which were distinguished based on expression of genes associated with inflammatory responses, GM-CSF signaling and aurora B signaling. Subsequently, 61 single BM macrophages were captured from a patient with newly diagnosed SJIA and laboratory abnormalities consistent with early MAS. Expression profiles in this patient were broadly similar to control macrophages by PCA, and all three macrophage clusters were represented. However, a distinct subpopulation of BM macrophages (9 of 61 cells) from the SJIA/MAS patient was identified that exhibited markedly altered transcriptional profiles. Compared to other macrophages within the cluster, this SJIA/MAS macrophage population showed alterations in gene pathways including “endocytic vesicle membranes” (p=8.44E−14), “phagosome activity” (p=2.98e−9) and “vesicle-mediated transport” (p=1.05E−07) suggesting that the population hemophagocytic macrophages was correctly identified. Consistent with the observations in peripheral blood, this macrophage population had a strong IFN-induced signature (“cellular response to interferon gamma”, adjusted p=1.35e−14). These cells also showed a proinflammatory gene expression signature, including significant enrichment for genes regulated by NF-kB and STAT1 as well as several molecules that could contribute to increased responsiveness to IFN-γ. Further examination of gene expression profiles in this macrophage population also revealed perturbations in the aryl hydrocarbon receptor (AHR) signaling pathway. It has been recently demonstrated that AHR downregulation in SJIA skewed monocyte differentiation towards macrophage (rather than dendritic cell) phenotype possibly increasing the risk for MAS [67]. Together, these data show that SC RNA Seq profiling of BM macrophages can identify reproducible cellular clusters as well as potential biologically relevant subpopulations and pathways perturbed during inflammatory disorders that might be targetable in MAS.

Cellular sources of IFN-γ. Studies in animal models of HLH and translational studies in SJIA/MAS suggest that activation and expansion of macrophages in this clinical phenomenon are driven by IFN-γ derived from mainly CD817 cells [40,41]. CD8⁺ CD38^{high} CCR7⁻GranzymeB^{high} cytolyic effector phenotype T cells have been identified as the main source of IFN-γ in peripheral circulation in pHLH. Applicant observed expansion of the same population in MAS, but not in SJIA without MAS. This observation combined with MAS histology showing numerous infiltrating CD8+cells producing IFN-γ, suggest that CD8⁺ CD38^{high} CCR7GranzymeB^{high} effector phenotype T cells might be a prominent source of IFN-γ in MAS as well.

Considering the likely role of IFN-γ in hemophagocytic syndromes, these observations may explain unexpectedly high rates of MAS in SJIA treated with biologics. In the canakinumab trial, all episodes of MAS were triggered by infections, mainly viral illnesses [47]. In this scenario, an IFN-γ surge induced by infections could potentially trigger an exaggerated macrophage response eventually leading to MAS.

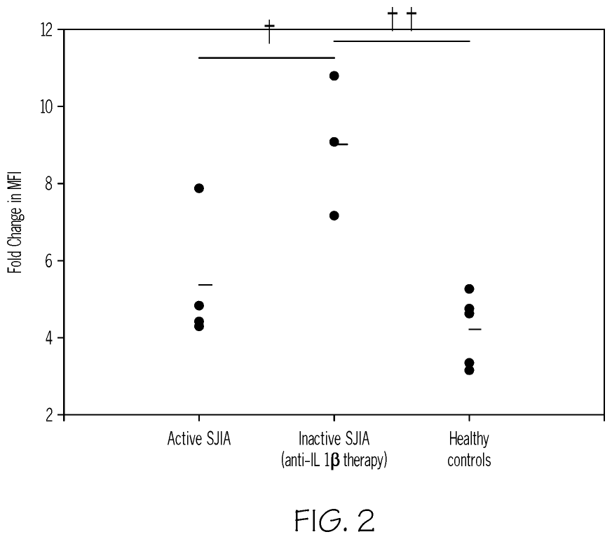

Applicant found that monocytes from SJIA patients stimulated with IFN-γ in vitro exhibited higher levels of phosphorylated STAT1 compared to monocytes from HCs. (FIG. 2). Unexpectedly, such hyper-responsiveness was even higher in SJIA patients treated with anakinra (n=3) despite the fact that the underlying SJIA in those patients was well controlled. Consistent with this observation, in the clinical trial of anakinra in SJIA, Quartier et al, reported a sustained increase in the strength of the IFN-induced signature in patients treated with anakinra (but not with placebo) regardless of the response status [12]. Applicant's analysis of the gene expression data generated in the clinical trial of another IL-1 inhibiting agent canakinumab [72] revealed similar results (not shown). The exact mechanisms leading to such hyper-responsiveness remain unknown, but increased expression of the positive regulator of IFN-γ signaling TRIM8 was observed, both in circulating monocytes and BM hemophagocytic macrophages.

### Example

Background: Systemic juvenile idiopathic arthritis (SJIA) is a severe and distinct subtype of childhood arthritis. Children with SJIA are at risk for macrophage activation syndrome (MAS), a life-threatening episode of hyperinflammation driven by interferon-gamma (IFNγ). Previous work has suggested that monocytes in SJIA display hyperresponsiveness to IFNγ, but the molecular basis of this has been unclear.

Objective: Utilize transcriptional profiling of monocytes and macrophages in SJIA to identify polarization phenotypes including features of interferon response

Methods: Bulk RNA-sequencing (RNA-seq) was performed on purified monocytes from 26 patients with SJIA without overt MAS. In addition, single-cell RNA-seq was performed on isolated bone marrow macrophages from control patients and patients with SJIA and MAS. THP-1 monocytic cells and primary human monocyte-derived macrophages (MDM) were transfected with TRIM8-specific or negative control small-interfering RNA prior to stimulation with IFNγ.

Results: RNA-seq of purified SJIA monocytes revealed marked transcriptional changes between cells from patients with high vs low serum ferritin levels. Pathway analysis demonstrated enriched upregulated gene ontology pathways including Response to External Stimulus (p=2.73×10−17), Defense Response (p=2.66×10−14) and Inflammatory Response (p=1.95×10−11). When comparing the SJIA monocyte signature to well-characterized polarization phenotypes, Applicant identified substantial overlap with multiple polarization states, most notably M1 and M2b, but little evidence of IFNγ-induced signature. Among the most highly upregulated genes in SJIA monocytes was tripartite motif containing 8 (TRIM8), an E3 ubiquitin-ligase involved in activation of IFNγ through promoting degradation of the suppressor of cytokine signaling 1 (SOCS1). Elevated TRIM8 expression was found in monocytes from both active and inactive SJIA patients, with the highest levels in those with subclinical MAS features (n=3). Furthermore, we utilized single cell RNA-seq to determine gene expression profiles of bone marrow macrophages from a patient with subclinical MAS. This identified a distinct subpopulation of bone marrow macrophages which exhibited markedly altered transcriptional profiles, with alterations in gene pathways predicted for hemophagocytes in MAS, including cellular response to interferon gamma (p=1.35e−14), endocytic vesicle membranes (p=8.44E−14), and phagosome (p=2.98e−9). These bone marrow macrophage also showed significantly increased TRIM8 expression (6.4-fold increase, p=0.02). To confirm the role of TRIM8 in augmenting macrophage responses to IFNγ, RNA interference was used to knock-down TRIM8 expression in THP-1 cells and MDM. Inhibitor treatment led to reduced levels of TRIM8 transcript lasting >48 hours. TRIM8 knock-down macrophages showed significant reductions in both early (4 hour) and late (24-48 hours) response to IFNγ, as determined by production of CXCL9, a biomarker for MAS activity in both humans and animal models.

Conclusions: Peripheral blood monocytes in SJIA display markers of multiple polarization states, while during MAS tissue macrophages demonstrate a clear IFNγ response phenotype. TRIM8 is highly expressed in both monocytes and macrophages in SJIA, and in vitro knockdown of TRIM8 impairs IFNγ responses in macrophages. Together these data provide a molecular mechanism for monocyte hyperresponsiveness to IFNγ in SJIA, as well as a novel therapeutic target for MAS.

### REFERENCES

1. Silverman E D, Miller J J, Bernstein B, Shafai T. Consumption coagulopathy associated with systemic juvenile rheumatoid arthritis. J Pediatr 1983; 103:872-6.
2. Hadchouel M, Prieur A M, Griscelli C. Acute hemorrhagic, hepatic, and neurologic manifestations in juvenile rheumatoid arthritis: possible relationship to drugs or infection. J Pediatr 1985; 106:561-66
3. Mouy R, Stephan J L, Pillet P, et al. 1996. Efficacy of cyclosporine A in the treatment of macrophage activation syndrome in juvenile arthritis: report of five cases. J Pediatr 1996; 129:750-54
4. Grom A A, Passo M. 1996. Macrophage activation syndrome in systemic juvenile rheumatoid arthritis. J Pediatr 129:630-32.
5. Ravelli A, De Benedetti F, Viola S, Martini A. 1996. Macrophage activation syndrome in systemic juvenile rheumatoid arthritis successfully treated with cyclosporine. J Pediatr 128:275-78.
6. Stephan J L, Kone-Paut I, Galambrun C, et al. 2001. Reactive haemophagocytic syndrome in children with inflammatory disorders. A retrospective study of 24 patients. Rheumatology 40:1285-92.
7. Sawhney S, Woo P, Murray K J. Macrophage activation syndrome: a potentially fatal complication of rheumatic disorders. Arch Dis Childhood 2001; 85:421-26.
8. Mellins E D, Macubas C, Grom A A. Pathogenesis of systemic juvenile idiopathic arthritis: some answers, more questions. Nature Reviews Rheumatology 2011; 7:416-26.
9. Rossi-Semerano L, and I Kone-Paut. Is Still's disease an autoinflammatory syndrome? Int J Inflamm 2012; 480373.
10. Rigante D and L Cantarini. The systemic onset variant of juvenile idiopathic arthritis needs to be recorded as an autoinnflammatory syndrome: comment on the review by Nigrovic. Arthritis Rheumatol 2014; 66; 2645
11. Pascual V, Allantaz F, Arce E, et al. Role of interleukin-1 (IL-1) in the pathogenesis of systemic onset juvenile idiopathic arthritis and clinical response to IL-1 blockade. J Exp Med 2005; 201:1479-86,
12. Quartier P, Allantaz F, Cimaz R, Pillet P, Messiaen C, Bardin C, et al. A multicentre, randomised, double-blind, placebo-controlled trial with the interleukin-1 receptor antagonist anakinra in patients with systemic-onset juvenile idiopathic arthritis. Ann Rheum Dis 2011; 70:747-54.
13. Ruperto N, Brunner H I, Quartier P, Constantin T, Wulfraat N, Horneff G, Brik R, McCann L, Nistala K, Wouters C, Cimaz R, Ferrandiz M A, Fato B, Grom A A, Magnusson, Ozen S, Abrams K, Kim D, Martini A, Lovell D J. Two Randomized Trials of Canakinumab in Systemic Juvenile Idiopathic Arthritis. New Engl J Med 2012; 367:2396-406.
14. De Benedetti F, and A Martini. Is systemic juvenile rheumatoid arthritis an IL-6 mediated disease. J Rheumatol 1998; 25:203-7.
15. Yokota S, Imagawa T, Mori M, et al. Efficacy and safety of tocilizumab in patients with systemic-onset juvenile idiopathic arthritis: a randomised, double-blind, placebo-controlled, withdrawal phase III trial. Lancet 2008; 371: 998-1006.
16. De Benedetti F, Brunner H I, Ruperto N, Kenwright, A, Ravelli A, Schneider, Woo P, Wouters C, Zemel, L, Burgos-Vargos R, Dolezalova P, Grom A A, Wulffraat N, Zuber Z, Zulian F, Lovell D and Martini A. Randomized Trial of Tocilizumab in Systemic Juvenile Idiopathic Arthritis. New Engl J Med 2012; 367:2385-95.
17. Grom A A. N K dysfunction: a common pathway in systemic onset juvenile rheumatoid arthritis, macrophage activation syndrome, and hemophagocytic lymphohistiocytosis. Arthritis Rheum 2004; 50:689-698.
18. Chandrakasan S, Filipovich A H. Hemophagocytic lymphohistiocytosis: advances in pathophysiology, diagnosis, and treatment. J Pediatr 2013; 163:1253-9.
19. Favara B E, Feller A C, Pauli M, et al. 1997. Contemporary classification of histiocytic disorders. The WHO Committee on Histiocytic/Reticulum Cell Proliferations. Reclassification Working Group of the Histiocyte Society. Med Pediatr Oncol 199; 29:157-66.
20. Jordan M B, Allen C E, Weitzman S, et al. How I treat hemophagocytic lymphohistiocytosis. Blood 2011; 118:4041-52.
21. Henter J I, Home A, Arico M, Egeler R M, Filipovich A H, et al. HLH-2004: Diagnostic and therapeutic guidelines for hemophagocytic lymphohistiocytosis. Pediatr Blood Cancer 2007; 48:124-131.
22. Jenkins M R, Rudd-Schmidt J A, Lopez J A, Ramsbottom K M, Mannering S I, Andrews D M, Voskoboinik I, Trapani J A. Failed CTL/N K cell killing and cytokine hypersecretion are directly linked through prolonged synapse time. J Exp Med 2015; 212:307-17.
23. Menasche G, Feldmann J, Fischer A, de Saint Basile G. Primary hemophagocytic syndromes point to a direct link between lymphocyte cytotoxicity and homeostasis. Immunol Rev 2005; 203:165-179.
24. Kagi D, Odermatt B, Mak T W. Homeostatic regulation of CD8+ T cells by perforin. Eur J Immunol 1999; 29:3262-72.
25. Lykens J E, Terrell C E, Zoller E E, Risma K, Jordan M B. Perforin is a critical physiologic regulator of T-cell activation. Blood 2011; 118:618-626.
26. Stepp S E, Dufourcq-Lagelouse R, Le Deist F, et al. Perforin gene defects in familial hemophagocytic lymphohistiocytosis. Science 1999; 286:1957-59
27. Feldmann J, Callebaut I, Raposo G, et al. Munc13-4 is essential for cytolytic granules fusion and is mutated in a form of familial hemophagocytic lymphohistiocytosis (FHL3). Cell 2003; 115:461-73
28. zur Stadt U, Schmidt 5, Diler A S, et al. Linkage of familial hemophagocytic lymphohistiocytosis (FHL) type-4 to chromosome 6q24 and identification of mutations in syntaxin 11. Hum Mol Genet 2005; 14:827-34
29. zur Stadt U, Rohr J, Seifert W, et al. Familial hemophagocytic lymphohistiocytosis type 5 (FHL-5) is caused by mutations in Munc18-2 and impaired binding to Syntaxin 11. Am J Hum Genet 2009; 85:482-92.
30. Kogawa K, Lee S M, Villanueva J, et al. Perforin expression in cytotoxic lymphocytes from patients with hemophagocytic lymphohistiocytosis and their family members. Blood 2002; 99:61-6.
31. Zhang K J, Jordan M B, Marsh R, et al. 2011. Hypomorphic mutations in PRF1, MUNC13-4, and STXBP2 are associated with adult-onset familial HLH. Blood 2011:118; 5794-98.
32. Grom A A, Villanueva J, Lee S, et al. Natural killer cell dysfunction in patients with systemic-onset juvenile rheumatoid arthritis and macrophage activation syndrome. J Pediatr 2003; 142:292-96.
33. Vastert S J, van Wijk R, D'Urbano L E, et al. Mutations in the perforin gene can be linked to macrophage activation syndrome in patients with systemic onset juvenile idiopathic arthritis. Rheumatology 2010; 49:441-49.
34. Cifaldi L, Prencipe G, Caiello I, Bracaglia C, Strippoli R, De Benedetti F. Inhibition of natural killer cell cytotoxicity by interleukin-6: implications for the pathogenesis of macrophage activation syndrome. Arthritis & Rheumatol 2015.
35. Grom A A, Home A C, and De Benedetti F. Macrophage activation syndrome in the era of biologic therapy: clues to pathogenesis and impact on diagnostic approaches. Nature Rev Rheumatol 2016.
36. Kaufman K M, Linghu B, Szustakowski J D, A Husami, Yang F, Zhang K, Filipovich A, Fall N, Harley J B, Nirmala N R, and Grom A A. Whole Exome Sequencing Reveals Overlap Between Macrophage Activation Syndrome in Systemic Juvenile Idiopathic Arthritis and Familial Hemophagocytic Lymphohistiocytosis Arthritis& Rheum 2014; 66:3486-95.
37. Bleesing J, Prada E, Siegel D M, Villanueva J, Olson J, Ilowite N, Brunner H I, Griffin T, Graham T B, Sherry D D, Passo M H, Filipovich A, and Grom A A. The diagnostic significance of soluble CD163 and soluble interleukin-2 receptor alpha-chain in macrophage activation syndrome and untreated new-onset systemic juvenile idiopathic arthritis. Arthritis Rheum 2007; 56:965-71.
38. Behrens E M, Beukelman T, Paessler M, and Cron R Q. Occult macrophage activation syndrome in patients with systemic juvenile idiopathic arthritis. J Rheumatol 2007; 34:1133-8.
39. Bracaglia C, Sieni E, Da Ros M, De Fusco C, Micalizzi C, Cetica, V, Ciambotti B, Coniglio M L, Insalaco A, De Benedetti F, Maurizio A. Mutations of familial hemophagocytic lymphohistiocytosis related genes and abnormalities of cytotoxicity function tests in patients with macrophage activation syndrome (MAS) occurring in systemic juvenile idiopathic arthritis. Pediatric Rheumatology 2014, 12(Suppl 1):P53
40. Billiau A D, Roskams T, Van Damme-Lombaerts R, et al. 2005. Macrophage activation syndrome: characteristic findings on liver biopsy illustrating the key role of activated, IFN-gamma-producing lymphocytes and IL-6- and TNF-alpha-producing macrophages. Blood 105:1648-51.
41. Jordan M B, Hildeman D, Kappler J, Marrack P. An animal model of hemophagocytic lymphohistiocytosis (HLH): CD8+ T cells and interferon gamma are essential for the disorder. Blood 2004; 104:735-43.
42. Pachlopnik Schmid J, Ho C H, Chretien F, et al. Neutralization of IFNgamma defeats haemophagocytosis in LCMV-infected perforin- and Rab27a-deficient mice. EMBO Mol Med 2009; 1:112-24.
43. Henter J I, Elinder G, Soder 0, et al. Hypercytokinemia in familial hemophagocytic lymphohistiocytosis. Blood 1991; 78:2918-22.
44. Takada H, Takahata Y, Nomura A et al. Increased serum levels of interferon-gamma-inducible protein 10 and monokine induced by gamma interferon in patients with haemophagocytic lymphohistiocytosis. Clin Exp lnnunol 2003; 133:448-53.
45. Ravelli A, Schneider R, Weitzman S, Devlin C, Daimaru K, Yokota S, Takei S, De Benedetti F. Macrophage activation syndrome in patients with systemic juvenile idiopathic arthritis treated with tocilizumab. Arthritis Rheumatol 2014; Vol 66(Suppl):83-84.
46. Yokota S, Itoh Y, Morio T, Sumitomo N, Daimaru K, Minota S. Macrophage activation syndrome in patients with systemic juvenile idiopathic arthritis under treatment with tocilizumab. J Rheumatol 2015; 42:712-22.
47. Grom A A, Ilowite N T, Pascual V, Brunner H I, Martinin A, Lovell D, Rupert( ) N, Leon K, Lheritier K, and Abrams K. Canakinumab in systemic juvenile idiopathic arthritis: impact on the rate and clinical presentation of macrophage activation syndrome. Arthritis Rheumatol 2016
48. Miettunen P M, Narendran A, Jayanthan A, et al. Successful treatment of severe paediatric rheumatic disease-associated macrophage activation syndrome with interleukin-1 inhibition following conventional immunosuppressive therapy: case series with 12 patients. Rheumatology 2011; 50:417-19
49. Durand M, Troyanov Y, Laflamme P, Gregoire G. Macrophage activation syndrome treated with anakinra. J Rheumatol 2010; 37:879-80
50. Nigrovic P A, Mannion M, Prince F H, et al. Anakinra as first-line disease-modifying therapy in systemic juvenile idiopathic arthritis: report of forty-six patients from an international multicenter series. Arthritis Rheum 2011; 63:545-55.
51. Ilowite N T, Prather K, Lokhnygina Y, Schanberg L E, Elder M, Milojevic D, Verbsky J W, Spalding S J, Kimura Y, Imundo L F, Punaro M G, Sherry D D, Tarvin S E, Zemel L S, Birmingham J D, Gottlieb B S, Miller M L, O'Neil K, Ruth N M, Wallace C A, Singer N G, Sandborg C l. Randomized, double-blind, placebo-controlled trial of the efficacy and safety of rilonacept in the treatment of systemic juvenile idiopathic arthritis. Arthritis Rheumatol. 2014; 66:2570-9.
52. Shimizu M, Yokoyama T, Yamada K, et al. Distinct cytokine profiles of systemic-onset juvenile idiopathic arthritis-associated macrophage activation syndrome with particular emphasis on the role of interleukin-18 in its pathogenesis. Rheumatology 2010; 49:1645-53.
53. Maeno N, Takei S, Imanaka H, et al. Increased interleukin-18 expression in bone marrow of a patient with systemic juvenile idiopathic arthritis and unrecognized macrophage-activation syndrome. Arthritis Rheum 2004; 50:1935-38.
54. Novick D, Elbirt D, Miller G, et al. High circulating levels of free interleukin-18 in patients with active SLE in the presence of elevated levels of interleukin-18 binding protein. Cytokine 2009; 48:103-4.
55. Favilli F, Anzilotti C, Martinelli L, et al. IL-18 activity in systemic lupus erythematosus. Ann N Y Acad Sci 2009; 1173:301-9.
56. Chiossone L, Audonnet S, Chetaille B, et al. Protection from inflammatory organ damage in a murine model of hemophagocytic lymphohistiocytosis using treatment with IL-18 binding protein. Front Immunol 2012; 3:239-249.
57. Sikora K A, Fall N, Thornton S, Grom A A. The limited role of interferon-gamma in systemic juvenile idiopathic arthritis cannot be explained by cellular hyporesponsiveness. Arthritis Rheum 2012:3799-808
58. Ogilvie E M, Khan A, Hubank M, et al. Specific gene expression profiles in systemic juvenile idiopathic arthritis. Arthritis Rheum 2007; 56:1954-65.
59. Fall N, Barnes M, Thornton S, Luyrink L, Olson J, Ilowite N T, Gottlieb E, Griffin T, Sherry D D, Thompson S Glass D N, Colbert R A, and Grom A A. Gene expression profiling of peripheral blood from patients with untreated new-onset systemic juvenile idiopathic arthritis reveals molecular heterogeneity that may predict macrophage activation syndrome. Arthritis Rheum 2007; 56:3793-804.
60. Allantaz F, Chaussabel D, Stichweh D, Bennett L, Allman W, Mejias A, Ardura M, Chung W, Smith E, Wise C, Palucka K, Ramilo 0, Punaro M, Banchereau J, Pascual V. Blood leukocyte microarrays to diagnose systemic onset juvenile idiopathic arthritis and follow the response to IL-1 blockade. J Exp Med 2007; 204:2131-44.
61. Bracaglia C, Kathy de Graaf K, Marafon D P, D'Ario G, Guilhot F, Ferlin W, Prencipe G, Caiello I, Davi D, Schulert G, Ravelli A, Grom A A, De Min, De Benedetti F. Elevated circulating levels of interferon-y and interferon-induced chemokines characterize patients with macrophage activation syndrome complicating systemic JIA. Ann Rheum Dis 2017; 76:166-172.
62. Toniato E, Chen X P, Losman J, Flati V, Donahue L, Rothman P. TRIM8/GERP RING finger protein interacts with SOCS-1. J Biol Chem 2002; 277:37315-22.
63. Olsson A, Venkatasubramanian M, Chaudhri V K, Aronow B J, Salomonis N, Singh H, Grimes H L. Single-cell analysis of mixed-lineage states leading to a binary cell fate choice. Nature. 2016; 537:698-702. PMID:27580035; PMCID:PMC5161694.
64. Schulert G S, Zhang, M, Fall N, Husami A, Kissell D, Hanosh A, Zhang K, Davis K, Jentzen J M, Siddiqui J, Smith L B, Harms P V V, Grom A A, and Cron R Q. Whole exome sequencing reveals mutations in hemophagocytic lymphohistiocytosis linked genes among fatal cases of H1N1 infection. J Infect. Dis 2016 PMID:26597256, PMCID:PMC4779301.
65. Macaubas C, Nguyen K D, Peck et al. Alternative activation in systemic juvenile idiopathic arthritis monocytes. Clin Immnol 2012; 142; 367-72.
66. Schwartz D M, Bonelli M, Gadina M, O'Shea J J. Type I/II cytokines, JAKs, and new strategies for treating autoimmune diseases. Schwartz D M, Bonelli M, Gadina M, O'Shea J J. Nat Rev Rheumatol 2016; 12:25-36.
67. Cepika A M, Banchereau R, Segura E, Ohouo M, Cantarel B, Goller K, Cantrell V, Ruchaud E, Gatewood E, Nguyen P, Gu J', Anguiano E, Zurawski S, Baisch J M, Punaro M, Baldwin N, Obermoser G, Palucka K, Banchereau J, Amigorena S, Pascual V. A multidimensional blood stimulation assay reveals immune alterations underlying systemic juvenile idiopathic arthritis. J Exp Med. 2017 Nov. 6; 214(11):3449-3466
68. Petty, R. E., et al., International League of Associations for Rheumatology classification of juvenile idiopathic arthritis: second revision, Edmonton, 2001. J Rheumatol, 2004. 31(2): p. 390-2.
69. Ravelli A, Minoia F, Davi S, Home A C, Bovis F, Pistorio P, Aric6 M, Avcin T, Behrens E M, De Benedetti F, Filipovic A H, Grom A A, Henter J I, llowite N T, Jordan M B, Khubchandani R, Kitoh T, Lehmberg K, Lovell D, Miettunen P, Nichols K, Ozen S, Pachlopnick J, Ramanan A V, Russo R, Schneider R, Sterba G, Uziel Y, Wallace W, Wouters C, Demirkaya E, Brunner H I, Martini A, Ruperto N, and R Q Cron, on behalf of the Pediatric Rheumatology International Trials Organization, the Childhood Arthritis & Rheumatology Research Alliance, the Pediatric Rheumatology Collaborative Study Group, and the Histiocyte Society. Development and initial validation of classification criteria for macrophage activation syndrome complicating systemic juvenile idiopathic arthritis. Arthritis Rheumatol 2016; 68:566-76. PMID:26314788.
70. Wallace, C. A., et al., Preliminary criteria for clinical remission for select categories of juvenile idiopathic arthritis. J Rheumatol, 2004; 31:2290-4.
71. Dinarello C A. Interleukin-18 and the pathogenesis of inflammatory diseases. Semin Nephrol 2007; 27:98-114.
72. Brachat, A H, Grom A A, VVulffraat N, Brunner H I, Quartier P, Brik R, McCann L, Ozdogan H, Rutkowska-Sak L, Schneider R, Gerloni V, Harel L, Terreri M, Houghton K, Joos R, Kingsbury D, Lopez-Benitez J M, Bek S, Schumacher M, Valentin M A, Gram H, Abrams K, Martini A, Lovell D J, Nirmala N R, Ruperto N. Early Changes in Gene Expression and Inflammatory Proteins in Systemic Juvenile Idiopathic Arthritis Patients on Canakinumab Therapy. Arthr Res Ther 2017. PMID:28115015, PMCID:PMC5260050
73. Suzuki T, Mayhew C, Sallese A, Chalk C, Carey B C, Malik P, Wood R E, Trapnell B C. Use of induced pluripotent stem cells to recapitulate pulmonary alveolar proteinosis pathogenesis. Am J Respir Crit Care Med. 2014; 189:183-93.
74. Hatakeyama S. TRIM Family Proteins: Roles in Autophagy, Immunity, and Carcinogenesis. 2017; 42:297-311.
75. Toniato E, Chen X P, Losman J, Flati V, Donahue L, Rothman P. TRIM8/GERP RING finger protein interacts with SOCS-1. J Biol Chem 2002; 277:37315-22.
76. Okumura F, Matsunaga Y, Katayama Y, Nakayama K I, Hatakeyama S. TRIM8 modulates STAT3 activity through negative regulation of PIAS3. J Cell Sci. 2010; 123:2238-45.
77. Li Q, Yan J, Mao A P, Li C, Ran Y, Shu H B, Wang Y Y. Tripartite motif 8 (TRIM8) modulates TNFa- and IL-113-triggered N F-K B activation by targeting TAK1 for K63-linked polyubiquitination. Proc Natl Acad Sci USA 2011; 108:19341-6.
78. Murray P J, Allen J E, Biswas S K, et al. Macrophage activation and polarization: nomenclature and experimental guidelines. Immunity 2014; 41:14-20.
79. Schulert G S, Fall N, Harley J B, Shen N, Lovell D J, Thornton S, Grom A A. Monocyte MicroRNA Expression in Active Systemic Juvenile Idiopathic Arthritis Implicates MicroRNA-125a-5p in Polarized Monocyte Phenotypes. Arthritis Rheumatol 2016; 68:2300-13.
80. Do T, Tan R, Bennett M, Medvedovic M, Grom A A, Shen N, Thornton S, and Schulert G S. MicroRNA networks associated with systemic JIA regulate CD163 expression and function in macrophages through distinct mechanisms. J Leukoc Biol. 2018 January; 103(1):71-85. doi: 10.1002/JLB.2A0317-107R. Epub 2017 Dec. 29. PMID:29345059.
81. Barnes M G, Grom A A, Thompson S D, Ilowite N T, Olson J C, Sherry D D, Gottlieb B S, Aronow B J, Pavlidis P, Hinze C, Thornton S, Griffin T A, Colbert C A, and D N Glass. Subtype-specific peripheral blood gene expression profiles in new onset untreated juvenile idiopathic arthritis. Arthritis Rheum 2009; 60:2102-12. PMID: 19565513 PMCID:PMC2782469

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method of identifying and treating an individual as having or being high risk for Macrophage Activation Syndrome (MAS), comprising the steps of
a. detecting a tripartite motif 8 (TRIM8) protein level or a tripartite motif 8 (TRIM8) mRNA level in a biological sample obtained from said individual; and
b. providing a treatment to said individual;
wherein if said TRIM8 protein level or said mRNA level is increased compared to a control value, said individual is identified as being high risk for MAS and said treatment comprises administering an immunosuppressive therapy; and
wherein if said TRIM8 protein or mRNA level is not increased or is the same as compared to said control value, said individual is not identified as being at high risk for MAS and said treatment does not comprise administration with immunosuppressive therapy.

2. The method of claim 1, wherein said individual is diagnosed or suspected of having systemic juvenile idiopathic arthritis (SJIA).

3. The method of claim 1, wherein said individual is diagnosed with a viral illness.

4. The method of claim 1, wherein said viral illness is selected from H1N1, Dengue fever, adenovirus, EBV, CMV, or a combination thereof, wherein a determination of elevated TRIM8 protein or mRNA compared to a control value in said individual indicates that said individual is at increased risk of developing MAS, wherein said individual having an increased risk of developing MAS is administered an agent an immunosuppressive therapy wherein said administration comprises an increased dose compared to a recommended amount, a recommended frequency of administration compared to a recommended amount, or a combination thereof.

5. The method of claim 1, wherein said step a) comprises detecting TRIM8 mRNA.

6. The method of claim 1, wherein said step a) comprises detecting TRIM8 protein.

7. The method of claim 1, wherein said biological sample is blood from said individual.

8. The method of claim 1, wherein said immunosuppressive therapy is selected from a JAK/STAT inhibitor, an anti-interferon gamma agent, an increased dosage of an existing immunosuppressive regime, or a combination thereof.

9. The method of claim 1, wherein said immunosuppressive therapy is an immunomodulator selected from tofacitimib, ruxolitinib, baricitinib, and oclacitinib.

10. The method of claim 1, wherein said immunosuppressive therapy is an immunomodulator selected from INCB0391 10, AZD1480, fedratinib, AT9283, AG-490, momelotinib, WP1066, TG101209, gandotinib, NVP-BSK805, AZ 960, CEP-33779, Pacritinib, WHI-P154, XL019, S-Ruxolitinib, ZM 39923, Decernotinib, Cerdulatinib, filgotinib, FLLL32, BMS-91 1543, peficitinib, GLPG0634, a GLPG0634 analogue, Go6976, curcumol, cucurbitacin, lestaurtinib, upadacitinib, CHZ868, Solcitinib (GSK 2586184), NS-018; etanercept, infliximab, adalimumab, tocilizumab, rituximab, ofatumumab, belimumab, epratuzumab, abatacept, golimumab, certolizumab pegol, sifalimumab, anakinra, canakinumab, rilonacept, pf-04965842, 5-((2-((4-fluoro-3-methoxy-5-methylphenyl)amino)-5-methylpyrimidin-4-yl)amino)benzo[d]oxazol-2(3H)-one, disodium (5-((2-((4-fluoro-3-methoxy-5-methylphenyl)amino)-5-methylpyrimidin-4-yl)amino)-2-oxobenzo[d]oxazol-3(2H)-yl)methyl phosphate, tofacitinib, TG101348, Janex 1, PF-956980, WHI-P154, ZM-39923, NSC114792, PF-06263276, CEP-33779, AZD-1480, SHR0302, oclacitinib, PF-04965842, N-(3-acetamido-5-(quinaxalin-2-yl) phenyl) acrylamide, 1-[(2S,5R)-2-Methyl-5-(7H-pyrrolo[2,3-d]pyrimidin-4-ylamino)-1-piperidinyl]-2-propen-1-one malonate (PF-06651600), [(1S)-2,2-Difluorocyclopropyl][3-[2-[(1-methyl-1H-pyrazol-4-yl)amino]-4-pyrimidinyl]-3,8-diazabicyclo[3.2.1]oct-8-yl]-methanone tosylate, emapalumab, azathioprine, cyclophosphamide, cyclosporine, hydroxychloroquine, leflunomide, methotrexate, mycophenolate, sulfasalazine, apremilast, tofacitinib, azathioprine, mercaptopurine, steroids, cortisone, cortisone acetate, dexamethasone, hydrocortisone, hydrocortisone acetate, methylprednisolone, prednisolone, prednisone, tixocortol pivalate, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, acleometasone dipropionate, betamethasone valerate, betamethasone dippropionate, prednicarbate, clobetasone-17-butyrate, clobetasol-17-propionate, fluocortilone caproate, fluocortolone pivalate, and fluprednidene acetate, hydrocortisone-17-butyrate, 17-aceponate, 17-buteprate, and prednicarbate.

11. The method of claim 1, wherein said immunosuppressive therapy is tofacitinib.

12. The method of claim 1, wherein said individual is identified as being high risk for MAS and said immunosuppressive therapy comprises administration of an amount of immunosuppressive agent that is increased as compared to a recommended dose.

13. A method of treating an individual having Macrophage Activation Syndrome (MAS) or suspected of being high risk for developing Macrophage Activation Syndrome (MAS), comprising administering an immunosuppressive therapy selected from a JAK/STAT inhibitor, an anti-interferon gamma agent, or a combination thereof.

14. The method of claim 13, wherein said individual is diagnosed with systemic juvenile idiopathic arthritis (SJIA).

15. The method of claim 13, wherein said viral illness is selected from H1N1, Dengue fever, adenovirus, EBV, CMV, or a combination thereof, wherein a determination of elevated TRIM8 protein or mRNA compared to a control value in said individual indicates that said individual is at increased risk of developing MAS, wherein said individual having an increased risk of developing MAS is administered an immunosuppressive therapy.

16. The method of claim 13, wherein said individual is characterized as high risk for Macrophage Activation Syndrome (MAS) based on an elevated tripartite motif 8 protein (TRIM8) protein or mRNA level in said individual as compared to a predetermined control value.

17. The method of claim 13, wherein said predetermined control value is an average TRIM8 protein or average TRIM8 mRNA level as found in an individual not having a viral illness or systemic juvenile idiopathic arthritis (SJIA).

18. The method of claim 13, wherein said immunosuppressive therapy is tofacitinib.
